(19) United States
(12) Patent Application Publication
(10) Pub. No.: US 2003/0147974 A1
(43) Pub. Date: Aug. 7, 2003

(54) Process for obtaining crystalline rosemary acid

(75) Inventors: Markus Sauter, Gensingen (DE); Carsten Bender, Ummendorf (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma GmbH & Co. KG, Ingelheim (DE)

(21) Appl. No.: 10/356,309

(22) Filed: Jan. 31, 2003

Related U.S. Application Data
(60) Provisional application No. 60/386,687, filed on Jun. 6, 2002.

(30) Foreign Application Priority Data
Feb. 2, 2002 (DE) 10204270.5

(51) Int. Cl. A61K 35/78

(52) U.S. Cl. 424/725; 424/745

(57) ABSTRACT

The invention relates to a process for obtaining crystalline rosemary acid from balm, in which the ground-up plant parts are first extracted with an alcohol.

### RELATED APPLICATIONS

Benefit of U.S. Provisional Appliation Serial No. 60/386,687, filed on Jun. 6, 2002 is hereby claimed, and said Application is herein incorporated by reference.

### DESCRIPTION

The invention relates to a process for obtaining crystalline rosemary acid from balm, in which the ground-up plant parts are first extracted with an alcohol.

### BACKGROUND OF THE INVENTION

Rosemary acid is 3,4-dihydroxy-α-[[3-(3,4.dihydroxyphenyl)-1-oxo-2-propenyl]-oxy]-phenylpropionic acid of formula

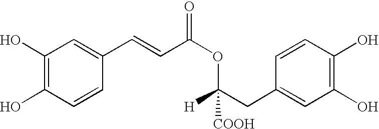

Rosemary acid has anti-inflammatory (e.g. DE 29 52 114.0) and anti-oxidative (e.g. WO 00/039248) properties. Consequently, rosemary acid is in great demand.

The processes for isolating rosemary acid known up till now have a number of drawbacks:

According to the process for isolating rosemary acid from Rosmarinus officinalis or Cichorium intybus described by Scarpati et al., Ricera Sci. 1958, 28, 2392-2393, the aqueous extracts of these plants have to be treated with lead salts and the lead compounds formed then have to be decomposed with hydrogen sulphide.

In the process described by Gestirner et al., Sci. Pharm. 1969, 37, 40-47, first of all fats have to be removed from the plants by repeated extraction with petroleum ether.

In the processes described in U.S. Pat. No. 4,354,035 or German Patent DE 32 34 312, balm (Melissa officinalis) has to be extracted once or several times with a large amount of hot water (10 to 30 times the amount at 80 to 100° C.). Then the aqueous extracts are highly concentrated (DE 32 34 312) (to 1/25 of the original volume), leading to an enormous energy demand, or directly acidified and extracted with water-insoluble organic solvents.

The aim of the present invention was therefore to provide an improved process for obtaining crystalline rosemary acid from balm on an industrial scale which avoids the disadvantages of the known methods.

### DETAILED DESCRIPTION OF THE INVENTION

Surprisingly it has been found that crystalline rosemary acid can be obtained from balm if ground-up plant material is extracted with an alcohol, the concentrated extract is taken up in water, the aqueous phase is extracted with ethers at a low pH and the rosemary acid is isolated from the ether extracts thus obtained.

The present invention thus relates to a process for obtaining crystalline rosemary acid from balm, in which the following steps are carried out successively:

(i) extracting ground-up plant material with an alcohol and concentrating the alcoholic extract,

(ii) taking up the residue obtained in water,

(iii) extracting the aqueous phase with one or more nonpolar solvents at a pH>4.4;

(iv) acidifying the aqueous phase to a pH between 3.5 and 4.3 and extracting the aqueous phase with an ether;

(v) concentrating the ethereal extract;

(vi) isolating and purifying the rosemary acid from the residue.

Balm leaves (Melissa officinalis) have proved to be a particularly suitable plant material.

Suitable alcohols for extracting the plant parts are generally aliphatic alcohols with 1 to 4 carbon atoms, particularly methanol, ethanol or isopropanol or mixtures thereof, most preferably methanol.

Suitable nonpolar solvents for extracting the aqueous phase at a pH>4.4 are generally aliphatic, cycloaliphatic or aromatic hydrocarbons or ethers or mixtures thereof, preferably aliphatic hydrocarbons with 5 to 8 carbon atoms, particularly pentane, hexane or heptane, most preferably n-hexane, cycloaliphatic hydrocarbons with 5 to 8 carbon atoms, particularly cyclopentane, cyclohexane, methylcyclohexane, or aromatic hydrocarbons with 6 to 9 carbon atoms, particularly toluene or xylene or aliphatic or alicyclic ethers such as for example diethyl ether, diisopropylether, methyl-tert-butylether, dioxane or tetrahydrofuran, while it is most particularly preferred to use toluene and methyl-tert-butylether in succession.

The aqueous phase is generally acidified using organic and inorganic acids, preferably aliphatic carboxylic acids such as for example formic acid, acetic acid, oxalic acid or trifluoroacetic acid or mineral acids such as for example phosphoric acid, nitric acid, hydrochloric acid or sulphuric acid, particularly hydrochloric acid, most preferably in the form of a dilute aqueous solution (1 to 4 normal).

Suitable ethers for extracting the acidified aqueous phase are generally aliphatic or alicyclic ethers such as for example diethyl ether, diisopropylether, methyl-tert-butylether, dioxane or tetrahydrofuran, most preferably methyl-tert-butylether.

Preferred embodiments of the invention are:

(A) processes in which in step (i) ground balm leaves are extracted twice with in each case 3 to 10 times, preferably 5 to 8 times the amount of methanol at boiling temperature.

(B) processes in which in step (ii) the residue is taken up in 2 to 8 times, preferably 4 to 6 times the amount of water. In the event that hereinabove or hereinbelow the relation of two compounds is indicated in the form “x to y fold amount of the first compound”, wherein x and y represent the lower and upper limit of said amount, this indication relates to “x to y” parts per weight of said first compound with respect to 1 part per weight of the second compound.

(C) processes in which in step (iii) the aqueous phase is extracted several times with an aromatic hydrocarbon, particularly toluene, and then at a pH of about 4.5 with an ether, particularly methyl-tert-butylether.

(D) processes in which in step (iv) after being acidified to a pH between 3.8 and 4.3 the aqueous phase is extracted several times with an ether selected from among diethyl ether, diisopropylether, methyl-tert-butylether, dioxane and tetrahydrofuran, particularly with methyl-tert-butylether.

(E) processes in which in step (vi) the residue obtained in step (v) is taken up in 1 to 5 times as much water, clarified with activated charcoal, the resulting mixture is filtered, the filtrate is inoculated with crystalline rosemary acid, cooled to temperatures of −10 to +10° C., preferably 0 to +5° C., and the precipitate is separated off.

In a most particularly preferred embodiment of the process according to the invention, balm leaves (Melissa officinalis) are refluxed with 6 to 10 times, preferably 7 to 9 times as much methanol for 2 to 6 hours, preferably 3 to 5 hours. After filtration the balm is again refluxed with 4 to 8 times as much fresh methanol for 0.5 to 3 hours. After filtering off the combined methanolic extracts are evaporated down.

The crude product obtained is distributed between 3 to 8 times as much toluene and 1 to 6 times as much water. The aqueous phase is extracted several times with toluene. The aqueous phase is extracted once or twice with methyl-tert-butylether (MTB-ether) at a pH of about 4.5. This organic phase is discarded. The aqueous phase is adjusted with 2N hydrochloric acid to a pH of 3.5-4.3 and extracted several times with MTB-ether. After each extraction the pH is adjusted again to 3.5-4.3 with 2N hydrochloric acid. The combined MTB phases are evaporated to dryness in vacuo.

The crude product is dissolved in water and combined with activated charcoal. After 15 minutes' stirring at about 50° C. the activated charcoal is filtered off and the filtrate is inoculated with rosemary acid. At −10 to +10° C. the mixture is stirred for about 6 hours and then overnight at ambient temperature. The suspension is cooled to 5° C. and suction filtered and washed to some extent with cold water. The residue is dried in vacuo at 40 to 80° C.

The following Example serves to illustrate a process for obtaining rosemary acid which is carried out by way of example. It is intended solely as a possible procedure provided as an illustration, without restricting the invention to its contents.

### EXAMPLE

2583 g of balm leaves (Melissa officinalis) are decocted by refluxing with 20 l of methanol for 4 hours. After filtration the balm is again refluxed with 14 l of fresh methanol for 2 hours. After filtering the combined methanolic extracts are evaporated down in vacuo. 370 g of crude product are obtained.

The crude product is distributed between 2 l of toluene and 1.5 l of water. The aqueous phase is extracted 5 times with 1 l of toluene. Then the aqueous phase is extracted twice at a pH of about 4.5 with 500 ml of methyl-tert-butylether (MTB-ether). This organic phase is discarded. The aqueous phase is adjusted to a pH of 3.5-4 with 2N hydrochloric acid and extracted 5 times with 1000 ml of MTB-ether. After each extraction the pH is readjusted to 3.5-4 with 2N hydrochloric acid. The combined MTB-phases are evaporated to dryness in vacuo. 59 g of foamy crude product remain.

The crude product is dissolved in 240 ml of water and combined with 5 g of activated charcoal. After 15 minutes' stirring at 50° C. the activated charcoal is filtered off and the filtrate is inoculated with rosemary acid. The mixture is stirred for 6 hours at 0-5° C. is and then overnight at ambient temperature. The suspension is cooled to 5° C. and suction filtered and washed to some extent with cold water. The residue is dried in vacuo at 60° C. for 24 hours. 27.8 g (1.08% based on the balm used) of crystalline rosemary acid are obtained (HPLC: 90.6% against standard), with a melting point of 162-164° C. and [α]D=99.7° (c=1.2 in ethanol).

### DESCRIPTION

The invention relates to a process for obtaining crystalline rosemary acid from balm, in which the ground-up plant parts are first extracted with an alcohol.

### BACKGROUND TO THE INVENTION

Rosemary acid is 3,4-dihydroxy-α-[[3-(3,4.dihydroxyphenyl)-1-oxo-2-propenyl]-oxy]-phenylpropionic acid of formula

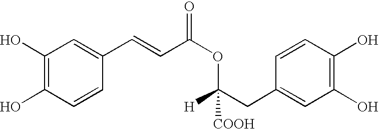

Rosemary acid has anti-inflammatory (e.g. DE 29 52 114.0) and anti-oxidative (e.g. WO 00/039248) properties. Consequently, rosemary acid is in great demand.

The processes for isolating rosemary acid known up till now have a number of drawbacks:

According to the process for isolating rosemary acid from Rosmarinus officinalis or Cichorium intybus described by Scarpati et al., Ricera Sci. 1958, 28, 2392-2393, the aqueous extracts of these plants have to be treated with lead salts and the lead compounds formed then have to be decomposed with hydrogen sulphide.

In the process described by Gestirner et al., Sci. Pharm. 1969, 37, 40-47, first of all fats have to be removed from the plants by repeated extraction with petroleum ether.

In the processes described in U.S. Pat. No. 4,354,035 or German Patent DE 32 34 312, balm (Melissa officinalis) has to be extracted once or several times with a large amount of hot water (10 to 30 times the amount at 80 to 100° C.). Then the aqueous extracts are highly concentrated (DE 32 34 312) (to 1/25 of the original volume), leading to an enormous energy demand, or directly acidified and extracted with water-insoluble organic solvents.

The aim of the present invention was therefore to provide an improved process for obtaining crystalline rosemary acid from balm on an industrial scale which avoids the disadvantages of the known methods.

### DETAILED DESCRIPTION OF THE INVENTION

Surprisingly it has been found that crystalline rosemary acid can be obtained from balm if ground-up plant material is extracted with an alcohol, the concentrated extract is taken up in water, the aqueous phase is extracted with ethers at a low pH and the rosemary acid is isolated from the ether extracts thus obtained.

The present invention thus relates to a process for obtaining crystalline rosemary acid from balm, in which the following steps are carried out successively:

(i) extracting ground-up plant material with an alcohol and concentrating the alcoholic extract,

(ii) taking up the residue obtained in water,

(iii) extracting the aqueous phase with one or more nonpolar solvents at a pH>4.4;

(iv) acidifying the aqueous phase to a pH between 3.5 and 4.3 and extracting the aqueous phase with an ether;

(v) concentrating the ethereal extract;

(vi) isolating and purifying the rosemary acid from the residue.

Balm leaves (Melissa officinalis) have proved to be a particularly suitable plant material.

Suitable alcohols for extracting the plant parts are generally aliphatic alcohols with 1 to 4 carbon atoms, particularly methanol, ethanol or isopropanol or mixtures thereof, most preferably methanol.

Suitable nonpolar solvents for extracting the aqueous phase at a pH>4.4 are generally aliphatic, cycloaliphatic or aromatic hydrocarbons or ethers or mixtures thereof, preferably aliphatic hydrocarbons with 5 to 8 carbon atoms, particularly pentane, hexane or heptane, most preferably n-hexane, cycloaliphatic hydrocarbons with 5 to 8 carbon atoms, particularly cyclopentane, cyclohexane, methylcyclohexane, or aromatic hydrocarbons with 6 to 9 carbon atoms, particularly toluene or xylene or aliphatic or alicyclic ethers such as for example diethyl ether, diisopropylether, methyl-tert-butylether, dioxane or tetrahydrofuran, while it is most particularly preferred to use toluene and methyl-tert-butylether in succession.

The aqueous phase is generally acidified using organic and inorganic acids, preferably aliphatic carboxylic acids such as for example formic acid, acetic acid, oxalic acid or trifluoroacetic acid or mineral acids such as for example phosphoric acid, nitric acid, hydrochloric acid or sulphuric acid, particularly hydrochloric acid, most preferably in the form of a dilute aqueous solution (1 to 4 normal).

Suitable ethers for extracting the acidified aqueous phase are generally aliphatic or alicyclic ethers such as for example diethyl ether, diisopropylether, methyl-tert-butylether, dioxane or tetrahydrofuran, most preferably methyl-tert-butylether.

Preferred embodiments of the invention are:

(A) processes in which in step (i) ground balm leaves are extracted twice with in each case 3 to 10 times, preferably 5 to 8 times the amount of methanol at boiling temperature.

(B) processes in which in step (ii) the residue is taken up in 2 to 8 times, preferably 4 to 6 times the amount of water.

(C) processes in which in step (iii) the aqueous phase is extracted several times with an aromatic hydrocarbon, particularly toluene, and then at a pH of about 4.5 with an ether, particularly methyl-tert-butylether.

(D) processes in which in step (iv) after being acidified to a pH between 3.8 and 4.3 the aqueous phase is extracted several times with an ether selected from among diethyl ether, diisopropylether, methyl-tert-butylether, dioxane and tetrahydrofuran, particularly with methyl-tert-butylether.

(E) processes in which in step (vi) the residue obtained in step (v) is taken up in 1 to 5 times as much water, clarified with activated charcoal, the resulting mixture is filtered, the filtrate is inoculated with crystalline rosemary acid, cooled to temperatures of −10 to +10° C., preferably 0 to +5° C., and the precipitate is separated off.

In a most particularly preferred embodiment of the process according to the invention, balm leaves (Melissa officinalis) are refluxed with 6 to 10 times, preferably 7 to 9 times as much methanol for 2 to 6 hours, preferably 3 to 5 hours. After filtration the balm is again refluxed with 4 to 8 times as much fresh methanol for 0.5 to 3 hours. After filtering off the combined methanolic extracts are evaporated down.

The crude product obtained is distributed between 3 to 8 times as much toluene and 1 to 6 times as much water. The aqueous phase is extracted several times with toluene. The aqueous phase is extracted once or twice with methyl-tert-butylether (MTB-ether) at a pH of about 4.5. This organic phase is discarded. The aqueous phase is adjusted with 2N hydrochloric acid to a pH of 3.5-4.3 and extracted several times with MTB-ether. After each extraction the pH is adjusted again to 3.5-4.3 with 2N hydrochloric acid. The combined MTB phases are evaporated to dryness in vacuo.

The crude product is dissolved in water and combined with activated charcoal. After 15 minutes' stirring at about 50° C. the activated charcoal is filtered off and the filtrate is inoculated with rosemary acid. At −10 to +10° C. the mixture is stirred for about 6 hours and then overnight at ambient temperature. The suspension is cooled to 5° C. and suction filtered and washed to some extent with cold water. The residue is dried in vacuo at 40 to 80° C.

The following Example serves to illustrate a process for obtaining rosemary acid which is carried out by way of example. It is intended solely as a possible procedure provided as an illustration, without restricting the invention to its contents.

### EXAMPLE

2583 g of balm leaves (Melissa officinalis) are decocted by refluxing with 20 l of methanol for 4 hours. After filtration the balm is again refluxed with 14 l of fresh methanol for 2 hours. After filtering the combined methanolic extracts are evaporated down in vacuo. 370 g of crude product are obtained.

The crude product is distributed between 2 l of toluene and 1.5 l of water. The aqueous phase is extracted 5 times with 1 l of toluene. Then the aqueous phase is extracted twice at a pH of about 4.5 with 500 ml of methyl-tert-butylether (MTB-ether). This organic phase is discarded. The aqueous phase is adjusted to a pH of 3.5-4 with 2N hydrochloric acid and extracted 5 times with 1000 ml of MTB-ether. After each extraction the pH is readjusted to 3.5-4 with 2N hydrochloric acid. The combined MTB-phases are evaporated to dryness in vacuo. 59 g of foamy crude product remain.

The crude product is dissolved in 240 ml of water and combined with 5 g of activated charcoal. After 15 minutes' stirring at 50° C. the activated charcoal is filtered off and the filtrate is inoculated with rosemary acid. The mixture is stirred for 6 hours at 0-5° C. is and then overnight at ambient temperature. The suspension is cooled to 5° C. and suction filtered and washed to some extent with cold water. The residue is dried in vacuo at 60° C. for 24 hours. 27.8 g (1.08% based on the balm used) of crystalline rosemary acid are obtained (HPLC: 90.6% against standard), with a melting point of 162-164° C. and [α]D=99.7° (c=1.2 in ethanol).

We claim:

1. Process for obtaining crystalline rosemary acid from balm, wherein the following steps are carried out successively:
(i) extracting ground-up balm plant material with an alcohol and concentrating the alcoholic extract,
(ii) taking up the residue obtained in water,
(iii) extracting the aqueous phase with a nonpolar solvent at a pH>4.4;
(iv) acidifying the aqueous phase to a pH between 3.5 and 4.3 and extracting the aqueous phase with an ether;
(v) concentrating the ethereal extract;
(vi) isolating and purifying the rosemary acid from the residue.

2. Process according to claim 1, wherein step (i) ground balm leaves are extracted twice with in each case 3 to 10 times the amount of methanol at boiling temperature.

3. Process according to claim 1, wherein step (ii) the residue is taken up in 2 to 8 times the amount of water.

4. Process according to claim 1, wherein the aqueous phase in step (iii) is extracted several times with an aromatic hydrocarbon and at a pH of about 4.5 with an ether.

5. Process according to claim 1, wherein the aqueous phase in step (iv) after being acidified to a pH between 3.8 and 4.3 is extracted several times with an ether selected from among diethyl ether, diisopropylether, methyl-tert-butylether, dioxane and tetrahydrofuran.

6. Process according to claim 5, wherein the aqueous phase in step (iv) is extracted 2 to 8 times with methyl-tert-butylether [sic].

7. Process according to claim 1, wherein the residue in step (vi) obtained in step (v) is taken up in 1 to 5 times as much water, clarified with activated charcoal, the resulting mixture is filtered, the filtrate is inoculated with crystalline rosemary acid, cooled to temperatures of −10 to +10° C., preferably 0 to +5° C., and the precipitate is separated off.
